(19) United States
(12) Patent Application Publication
(10) Pub. No.: US 2011/0110888 A1
(43) Pub. Date: May 12, 2011

(54) Scaffold Coated and/or Impregnated with at Least One Bioactive Agent for Tissue Repair and Other Medical Applications

(76) Inventors: Hai-Qing Xian, Foster City, CA (US); Jian Q. Yao, Austin, TX (US); Hali Wang, The Hills, TX (US); Massoud Daheshia, Cedar Park, TX (US); Hui Liu, Austin, TX (US)

(21) Appl. No.: 13/001,284

(22) PCT Filed: Jun. 26, 2009
(86) PCT No.: PCT/US09/48885

§ 371 Date: Dec. 23, 2010

(30) Foreign Application Priority Data
Jun. 27, 2008 (US) 61076277

(51) Int. Cl. A61K 38/20; C12N 5/071; C12M 3/00; A61K 38/18; A61K 38/22; A61K 38/39; A61K 38/29; A61K 38/28; A61K 38/26; A61K 38/23; A61K 38/19; A61K 38/21; A61P 43/00

(52) U.S. Cl. 424/85.2; 435/395; 435/289.1; 514/7.6; 514/9.7; 514/8.8; 514/8.9; 514/9.1; 514/8.2; 514/8.5; 514/9.6; 514/8.1; 514/17.2; 514/9.3; 514/21.92; 514/11.8; 514/5.9; 514/11.7; 514/11.9; 424/85.1; 424/85.4; 514/7.7; 514/7.8

(57) ABSTRACT

A system and method for producing a scaffold coated and/or impregnated with at least one bioactive agent. This is accomplished by culturing at least one cell in the same medium as a scaffold, but without physical contact between the at least one cell and the scaffold. The system includes a container having a surface defining an interior compartment. A medium is disposed within the interior compartment. At least one cell is disposed within the medium, the at least one cell being capable of producing at least one bioactive agent. Further, a scaffold is disposed within the medium. The scaffold is physically separated from the at least one cell such that there is no contact between the scaffold and the at least one cell. Thus, as the at least one cell produces at least one bioactive agent, such as a growth factor, the at least one bioactive agent enters the medium, and contacts and coats and/or impregnates the scaffold. However, due to the physical separation of the scaffold and at least one cell, the at least one cell is prevented from contacting and coating and/or impregnating the scaffold. Thus, only the desired bioactive agent or agents coat and/or impregnate the scaffold.

### TECHNICAL FIELD

Medical systems, devices, and methods, more particularly, medical systems, devices, and methods for repair of tissues, such as tendons, ligaments, meniscus, cartilage, and other connective tissues.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Surgeries, such as reconstructive plastic and orthopedic surgeries, are used to treat patients with damaged tissues, such as tendons, ligaments, meniscus, cartilage, and other connective tissues. Such procedures may include transplantation (e.g., excising healthy host cartilage and reimplanting it where needed into the same patient). When using transplantation for repair of injured tissues, autologous tissue transplant is the gold standard. For example, following rupture of a tendon, autologous tissue may be used to “bridge” the two tendon ends. In particular, the tendon of the flexor hallucis longus (FHL) muscle of the leg has been used to bridge gaps in ruptured Achilles tendons. The FHL tendon is weaved through the ruptured Achilles tendon ends, and the distal end of the FHL tendon is adhered to the tendon of the flexor digitorum longus muscle of the second toe. However, there are drawbacks to transplantation using autologous tissue. First, the process requires not only surgery to repair the ruptured Achilles tendon, but also to obtain the FHL tendon. Further, use of the FHL tendon impairs the normal function of the FHL tendon and FHL muscle.

As another example, autologous chondrocyte cell transplantation has been used to repair cartilage defects for the purpose of reducing pain and restoring function. The success of this procedure may generally diminish over time, possibly due to reasons such as formation of fibrocartilage, inadequate development of repair tissue, poor cell differentiation, and/or poor bonding to the surrounding articular cartilage borders. Further, for autologous chondrocyte cell transplantation, two surgeries are required: chondrocytes are first obtained from an uninvolved area of cartilage and cultured for 14 to 21 days, then the cultured cells are injected into the defect exposed via an open incision and covered with a periosteal flap excised from the proximal medial tibia.

These and other problems with transplantation (e.g., amount of donor tissue available for transplantation is limited; and it is often not possible to form delicate three-dimensional implants from harvested tissue) are common to many transplant procedures. Thus, alternative methods of tissue repair have been explored. These include implantation of devices to promote healing of tissue.

For example, a scaffold, which can be used to promote differentiation and growth of cells, such as chondrocytes, can be implanted into a subject at the point of injury. These scaffolds may be coated with various bioactive agents, such as growth factors, and thus act as a delivery vehicle for those bioactive agents in order to enhance the healing process. Numerous synthetic or modified natural materials have been used as scaffolds, and include but are not limited to products containing hydroxyapatites, tricalcium phosphates, aliphatic polyesters, poly(lactic) acids (PLA), poly(glycolic)acids (PGA), polycaprolactone (PCL), cancellous bone allografts, human fibrin, plaster of Paris, apatite, wollastonite (calcium silicate), glass, ceramics, titanium, devitalized bone matrix, noncollagenous proteins, collagen and autolyzed antigen extracted allogenic bone. Further, hydrogels may serve as a scaffold, for applications in tissue engineering implantation. As one example, polyvinyl alcohol hydrogels can be modified with cell adhesion peptides. As another example, gradient hydrogels can be embedded with a peptide that can bind cell integrins (membrane bound receptors). Published U.S. Patent Application No. 2006/0233850 discloses scaffolds formed of hydrogels that are cross-linked in-situ in an infarcted region of the heart. Still other scaffolds may include mixtures of collagen and calcium phosphate.

The use of bioactive agents, such as growth factors, extracellular matrix (ECM) proteins, etc., to coat the scaffolds, improves clinical outcomes. Due to the coating, cells may attach, expand, differentiate and produce ECM on the scaffold in a desired way.

Currently, there are two general methods used to coat scaffolds with bioactive agents. In a first method, pure bioactive agents are obtained and used for scaffold coating. Such a method is described in Kim, H. D. et al., Bone Morphogenetic Protein 2-Coated Porous Poly-L-Lactic Acid Scaffolds: Release Kinetics and Induction of Pluripotent C3H10T1/2 Cells, Tissue Engineering, Vol. 4, No. 1, (1998), pp. 35-51. In that method, growth factors are secreted by a protein expression system (such as E. coli, yeast, or mammalian cells) and purified from culture medium. The purified growth factors are subsequently coated onto the scaffold. However, there are drawbacks to this method. For example, this method is very costly, since the purification process is very expensive.

A second approach is to culture cells on the scaffold, and keep cell lysates and secreted growth factors on the scaffold. Such a method is described in Freed, L. E. et al., Neocartilage Formation In Vitro and In Vivo Using Cells Cultured on Synthetic Biodegradable Polymers, J. of Biomedical Materials Research, Vol. 27 (1993), pp. 11-23. The shortcoming of this method is that cell debris may be present on the scaffold, in addition to the desired bioactive agents, thereby causing an immune response in the patient.

In view of the drawbacks associated with the complicated and expensive growth factor purification process and potential immune responses caused by cell debris, a new and/or improved system, device, and method for repairing tissues is desirable.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention provides a system for producing a scaffold that is coated and/or impregnated with bioactive agents. This system avoids the drawbacks described above in the Background, by culturing at least one cell in the same medium as a scaffold, but without physical contact between the at least one cell and the scaffold. More specifically, the system includes a container having a surface defining an interior compartment. A medium is disposed within the interior compartment. At least one cell is disposed within the medium, the at least one cell being capable of producing at least one bioactive agent. Further, a scaffold is disposed within the medium. The scaffold is physically separated from the at least one cell such that there is no contact between the scaffold and the at least one cell. Thus, as the at least one cell produces various bioactive agents, such as growth factors, the bioactive agents enter the medium, and contact and coat the scaffold. Further, as the scaffold may be porous, the bioactive agents may permeate the scaffold. However, due to the physical separation of the scaffold and at least one cell, the at least one cell is prevented from contacting the scaffold. Further, cell debris is also kept physically separate from the scaffold. Thus, only the desired bioactive agents coat and/or impregnate the scaffold. As a result, the scaffold is coated and/or impregnated in a manner that avoids the expensive process of purification of the bioactive agents, and the final coated and/or impregnated scaffold will not stimulate an immune response in the subject patient.

Another aspect of the present invention provides a device for repair of a tissue. The device includes at least one cell-produced bioactive agent, and a scaffold having affinity for the at least one cell-produced bioactive agent, wherein the scaffold has been cultured in the presence of, but absent direct physical contact with, the at least one cell-produced bioactive agent. Thus, as above, the scaffold is relatively inexpensive to prepare, and will not cause an immune response.

Another aspect of the present invention includes a method of coating and/or impregnating a scaffold with at least one bioactive agent. The method includes disposing at least one cell capable of producing at least one bioactive agent in medium in a container, positioning a scaffold in the medium, and maintaining the scaffold physically separate from the at least one cell while culturing the at least one cell in the medium.

Various features discussed below in relation to one or more of the exemplary embodiments may be incorporated into any of me above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE DRAWINGS

The accompanying drawings, which are incorporated in and constitute a part of this specification, illustrate exemplary embodiments of the invention and, together with the summary given above, and the detailed description given below, serve to explain the invention.

FIG. 1A is a schematic of a cell culture vessel including medium therein, adherent cells on a surface of the vessel, and a scaffold on a scaffold support structure, in accordance with the principles of the present invention.

FIG. 1B is a schematic of a cell culture vessel including a medium therein, adherent cells on a surface of the vessel, and a scaffold support structure with a scaffold thereon, the scaffold support structure surrounding the scaffold and including an orifice therein, in accordance with the principles of the present invention.

FIG. 1C is a schematic of a cell culture vessel including medium therein, adherent cells on a surface of the vessel, and a scaffold support structure with a scaffold thereon, the scaffold surrounded by a permeable membrane bag, in accordance with the principles of the present invention.

FIG. 1D is a schematic of a cell culture vessel including medium therein, adherent cells on a surface of the vessel, and a scaffold support structure with a scaffold thereon, and a chamber with apertures therethrough surrounding the scaffold, in accordance with the principles of the present invention.

FIG. 1E is a schematic of a bioreactor including medium therein, adherent cells on surfaces of the bioreactor, and multiple scaffold support structures including scaffolds thereon, the scaffolds being surrounded by bio active agent-permeable elements, in accordance with the principles of the present invention.

FIG. 2A is a schematic of a cell culture vessel including medium therein, a bioactive agent-permeable barrier dividing the vessel into a first compartment and a second compartment, with adherent cells located in the first compartment and scaffolds disposed in the second compartment, in accordance with the principles of the present invention.

FIG. 2B is a schematic of a cell culture vessel including medium therein, a bioactive agent-permeable barrier dividing the vessel into a first compartment and a second compartment, with nonadherent cells located in the first compartment and scaffolds disposed in the second compartment, in accordance with the principles of the present invention.

FIG. 3A is schematic of a cell culture vessel including medium therein, nonadherent cells disposed within the medium, and a scaffold support structure with a scaffold thereon, the scaffold being surrounded by a protein permeable bag, in accordance with the principles of the present invention.

FIG. 3B is schematic of a cell culture vessel including medium therein, nonadherent cells disposed within the medium, and a scaffold support structure with a scaffold thereon, the scaffold being surrounded by a chamber with holes, in accordance with the principles of the present invention.

FIG. 4 is a schematic of a bioreactor including multiple surfaces with scaffolds disposed thereon in medium, and nonadherent cells disposed in the medium, with the nonadherent cells surrounded by a bioactive agent-permeable member, in accordance with the principles of the present invention.

FIG. 5 is a schematic of a device including at least one cell-produced bioactive agent, and a scaffold having affinity for the at least one cell-produced bioactive agent, in accordance with the principles of the present invention.

### DETAILED DESCRIPTION

One or more specific embodiments of the present invention will be described below. In an effort to provide a concise description of these embodiments, not all features of an actual implementation may be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Referring to the Figures, one aspect of the present invention provides a system for producing a scaffold 10 that is coated and/or impregnated with at least one bioactive agent. This system avoids the drawbacks described above in the Background, in that at least one cell 12 is cultured in the same medium as a scaffold 10, but without contact between the at least one cell 12 and the scaffold 10. More specifically, the system includes a container 14 having a surface 16 defining an interior compartment 18. A medium 20 is disposed within the interior compartment 18. At least one cell 12 is disposed within the medium 20, the at least one cell 12 being capable of producing at least one bioactive agent 22. Further, a scaffold 10 is disposed within the medium 20. The scaffold 10 is physically separated from the at least one cell 12 such that there is no contact between the scaffold 10 and the at least one cell 12. Thus, as the at least one cell 12 produces various bioactive agents 22, such as growth factors, the bioactive agent or agents 22 enter the medium 20, and contact and coat the scaffold 10. Further, as the scaffold 10 may be porous, the bioactive agent or agents 22 may permeate the scaffold 10, thereby producing a scaffold 10 impregnated with at least one bioactive agent 22. However, due to the physical separation of the scaffold 10 and at least one cell 12, the at least one cell 12 is prevented from contacting the scaffold 10. Further, cell debris is also kept physically separate from the scaffold 10. Thus, only the desired bioactive agent or agents 22 coat and/or impregnate the scaffold 10. As a result, the scaffold 10 is coated and/or impregnated in a manner that avoids the expensive process of purification of the bioactive agents 22, and the scaffold 10 will not stimulate an immune response in the subject patient.

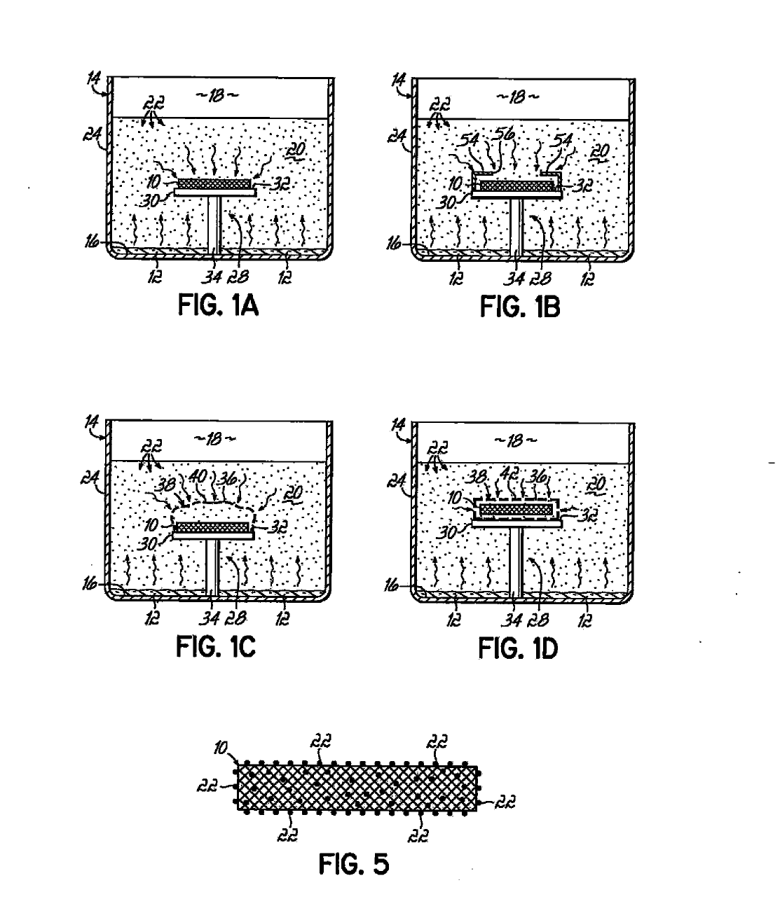

Thus, the system includes at least one cell 12, a container 14, medium 20, and a scaffold 10. As is known to those of ordinary skill in the art, cells in culture may include adherent cells (i.e., cells that adhere to a surface, such as a surface of a cell culture vessel) and nonadherent cells (i.e., cells that form a cell suspension within culture medium). Referring now to FIGS. 1A-1E, the at least one cell 12 of the illustrated embodiment is an adherent cell disposed on a surface 16 of the container 14. It will be recognized by those of ordinary skill in the art that the phrase “at least one cell” may encompass one cell or more than one cell, and thus, while the description and figures herein may refer to or show a cell, or a plurality of cells, such references are merely exemplary. In one embodiment, the source of the at least one cell 12 may be a primary cell culture secreting desired bioactive agents 22 (e.g., growth factors, ECM proteins, hormones, cytokines, chemokines, etc., or combinations thereof). In an alternate embodiment, the source of the at least one cell 12 may be an engineered cell or cells transfected with a gene of a desired bioactive agent 22 or a group of genes of desired bioactive agents 22. In such an alternate embodiment, the gene or genes may be integrated into the genome through viral or nonviral approaches, as are well known to those of ordinary skill in the art.

The container 14 may be chosen from a cell culture vessel 24 (as shown in FIGS. 1A-1D) and a bioreactor 26 (as shown in FIG. 1E). One example of a cell culture vessel 24 may be a cell culture dish. However, it will be recognized by those of ordinary skill in the art that the container 14 need not be chosen from a cell culture vessel 24 and bioreactor 26 (as these are merely exemplary), but may include any container suitable for culturing cells. Such containers are well known to those of ordinary skill in the art.

The medium 20 may be a culture medium used to support and grow the at least one cell 12. As is well known to those of ordinary skill in the art, such a medium 20 is a liquid or gel designed to support the growth of cells 12. Media used for cell culture may use specific cell types derived from plants or animals. Further, as is known to those of ordinary skill in the art, cells 12 grown in culture often cannot grow without the addition of, for instance, hormones or growth factors, which are present in vivo. And so, in the case of animal cells, this is often addressed by the addition of blood serum to the medium. Thus, a source of serum, such as horse or human serum, may be used. In yet another embodiment, one can use a defined medium 20 wherein the medium 20 is supplemented with specific factors. However, in the present invention, the medium 20 used during incubation may be serum free. One reason for the use of serum-free medium 20 may be due to the fact that serum may include undesirable bioactive agents that could be coated onto, and absorbed into, the scaffold 10. However, as described above, growth factors, etc. from serum are often needed, and so it would difficult to grow and expand the cells 12 without serum. This can be overcome in several ways. For example, in one embodiment, the cells 12 may be grown and expanded with FBS, washed extensively, then disposed on or in the container 14, and incubated for a short time (e.g., 24-48 hours) without serum.

Further, the at least one cell 12 must be induced to produce the desired bioactive agent 22. This is because resting cells would have very little production of bioactive agents (i.e., they would not secrete such bioactive agents e.g., growth factors—into the medium). Thus, the cells may be stimulated or induced when the cells are in the serum (for example, the last 1 to 2 hours of incubation time). If one does not want to induce the cells 12, then in an alternative embodiment, the cells 12 may be transfected with the gene of that specific, desired bioactive agent 22. The at least one cell 12 can produce different bioactive agents 22, and so if a specific bioactive agent 22 is desired, a barrier (such as a filter or membrane), with apertures 36 having specific diameters corresponding to the molecular weight of that factor, may be used to separate out the undesired bioactive agents 22 (for example, less than 20 KD to allow passage of specific bioactive agents). This will be discussed in greater detail below.

The illustrated embodiment of FIGS. 1A-1E also includes a scaffold 10 disposed in the interior compartment 18 of the container 14 and within the same medium 20 as the at least one cell 12. The scaffold 10 may be made from any materials known to those of ordinary skill in the art that are sufficient for being coated and/or impregnated with bioactive agents 22, such as growth factors, ECM proteins, hormones, cytokines, chemokines, etc., and is suitable for implantation into a subject body. Such materials may include, but are not limited to products containing hydroxyapatites, tricalcium phosphates, aliphatic polyesters, poly(lactic) acids (PLA), poly(glycolic)acids (PGA), polycaprolactone (PCL), cancellous bone allografts, human fibrin, plaster of Paris, apatite, wollastonite (calcium silicate), glass, ceramics, titanium, devitalized bone matrix, noncollagenous proteins, collagen, autolyzed antigen extracted allogenic bone, hydrogels, and mixtures of substances such as collagen and calcium phosphate. Further, while the description and figures refer to and show a single scaffold 10 (as in FIGS. 1A-1D) or multiple scaffolds 10 (as in FIG. 1E), the invention is not limited by the number of scaffolds.

Referring to the illustrated embodiments of FIGS. 1A-1E, when adherent cells are used, the scaffold 10 is not placed on the surface 16 of the container 14 to which the at least one cell 12 is adhered. Rather, to keep the scaffold 10 physically separated from the at least one cell 12, the system may include a scaffold support structure 28 positioned within the medium 20. In the illustrated embodiments, the scaffold support structure 28 includes a scaffold support 30 having a surface 32 on which the scaffold 10 is disposed, and a post 34. The particular configuration of the scaffold support structure 28 of the figures is merely exemplary, as any configuration that provides a surface for the scaffold while keeping the scaffold physically separated from the at least one cell will suffice. Thus, the scaffold support structure 28 provides a surface 32 for the scaffold 10 other than the surface 16 of the interior compartment 18 of the container 14. The scaffold 10 may then be disposed on the scaffold support structure 28. As can be seen in FIGS. 1A-1E, the scaffold 10 is kept physically separated from the at least one cell 12.

However, it will be recognized by those skilled in the art that a scaffold support structure is not necessary when adherent cells are used. For example, in an alternative embodiment (not shown), the at least one cell may be adhered to one surface of the container (and within the medium) while the scaffold is disposed on a different surface of the container (and within the medium). In this alternate embodiment, there are no cells adhered to the same surface as the scaffold. Of course, as the cells are cultured and expand, one must ensure that the cells do not contact the scaffold. In yet an alternative embodiment (not shown), the adherent cells may be on the same surface as the scaffold, as long as they do not contact the scaffold, and as long as they do not expand to contact the scaffold.

Thus, in the embodiment illustrated in FIGS. 1A-1E, at least one adherent cell 12 is cultured on a surface 16 of a container 14, such as the bottom of a cell culture dish or on the wall of a bioreactor 26. During the cell culture, the scaffold 10 (being a bioactive agent—affinitive scaffold) is immersed in the medium 20 physically separate from the bottom of the cell culture dish or the wall of the bioreactor 28. This may be done in multiple ways. Referring now to FIG. 1A, one way is to fix the scaffold 10 directly on the scaffold support structure 28 so that the bioactive agent 22, (e.g., growth factors) produced from the at least one cell 12 and into the medium 20 contacts and is coated on the scaffold 10. In such an embodiment, the scaffold support structure 28 is simply a solid shelf-type surface, as described above, with the scaffold 10 thereon being open to the medium 20. Alternatively, and as illustrated in FIG. 1B, the scaffold support structure 28 may substantially surround the scaffold 10. In this embodiment, the scaffold support structure 28 may be designed to retain the at least one bioactive agent 22, such as by including walls 54 that include or define an orifice 56 large enough to permit placement of the scaffold 10 and passage of the bioactive agent 22.

Another way of separating the scaffold 10 and at least one cell 12 is to include a barrier 38 between the at least one cell 12 and the scaffold 10, the barrier 38 being permeable to the at least one bioactive agent 22. This barrier 38 is not part of the scaffold support structure 28. In such embodiments, as shown in FIGS. 1C and 1D, the barrier 38 surrounds the scaffold 10. In these embodiments, the barrier 38 may be chosen from a membrane 40 and a chamber 42. For example, and referring to FIG. 1C, one may place the scaffold 10 in a membrane 40 on the scaffold support structure 28. The membrane 40 has many apertures 36 that are permeable to bioactive agents 22, but not to the at least one cell 12 or any cell debris. As described above, the at least one cell 12 can produce different bioactive agents 22, and so, if a specific bioactive agent is desired, a barrier 40 (such as a filter or membrane 40) with apertures 36 having specific diameters corresponding to the molecular weight of that factor, may be used to separate out the undesired bioactive agents 22 (for example, less than 20 kilo Dalton to allow passage of specific bioactive agents). Thus, the membrane 40 can be made of plastic or filter-like membranes. During cell culture, cell secreted growth factors or other desired bioactive agents 22 will be trapped or absorbed on the scaffold 10 in the culture system. Alternatively, and referring to FIG. 1D, the barrier 38 may be a chamber 42 that surrounds the scaffold 10. Like the membrane 40, the chamber 42 has many apertures 36 that are permeable to bioactive agents 22 of a certain size, but not to the at least one cell 12, or any cell debris.

The illustrated embodiment of FIG. 1E shows multiple scaffold support structures 28 including scaffolds 10 either on the supports 30 or on the supports 30 and surrounded by a barrier 38 (as described above with respect to FIGS. 1A-1D) in the environment of a bioreactor 26.

In certain embodiments, the at least one bioactive agent 22 may be chosen from a growth factor, an ECM protein, a hormone, a cytokine, a chemokine, and combinations thereof. However, this list is not limiting and is merely exemplary, as those skilled in the art will recognize that any desired bioactive agents that are produced by a cell may be used as the at least one bioactive agent of the present application, even if it is not a growth factor, ECM protein, hormone, cytokine, or chemokine.

When the at least one bioactive agent 22 is a growth factor, it may be, for example, bone morphogenetic protein (BMP), transforming growth factor (TGF), fibroblast growth factor (FGF), platelet-derived growth factor (PDGF), insulin-like growth factor (IGF), epidermal growth factor (EGF), vascular endothelial growth factor (VEGF), or combinations thereof. Growth factors, as known to those of ordinary skill in the art, are naturally occurring proteins capable of stimulating cell proliferation and differentiation.

BMPs are a group of growth factors and cytokines known for their ability to induce the formation of bone and cartilage. There are twenty such proteins. Of these, six of them (BMP2 through BMP7) belong to the TGF-β superfamily of proteins. BMPs interact with specific receptors on the cell surface, referred to as bone morphogenetic protein receptors (BMPRs). Signal transduction through BMPRs results in mobilization of members of the SMAD family of proteins. The signaling pathways involving BMPs, BMPRs and SMADs are important in the development of the heart, central nervous system, and cartilage, as well as post-natal bone development.

TGFs include those of the TGF-β family. TGF-β is a protein that comes in three isoforms: TGF-β1, TGF-β2 and TGF-β3. The TGF-β family includes inhibins, activin, hormone, bone morphogenetic protein, decapentaplegic and Vg-1. TGF-β controls proliferation, differentiation, and other functions of cells. It can also act as a negative autocrine growth factor.

FGFs are a family of growth factors involved in wound healing and embryonic development. The FGFs are heparin-binding proteins and interact with cell-surface associated heparan sulfate proteoglycans to assist in FGF signal transduction. FGF stimulates the proliferation of fibroblasts (a connective tissue cell) that give rise to granulation tissue, which fills up a wound space (i.e., the gap in a ruptured tendon) early in the wound healing process.

PDGF is a dimeric glycoprotein composed of two A or two B chains that regulates cell growth and division. In particular, it plays a role in blood vessel formation (angiogenesis), particularly the growth of blood vessels from already existing blood vessel tissue. Further, PDGF is an element in cellular division for fibroblasts.

IGFs are polypeptides with high sequence similarity to insulin. IGFs are part of a complex system that cells use to communicate with their physiologic environment. This complex system (often referred to as the IGF “axis”) consists of two cell-surface receptors (IGF1R and IGF2R), two ligands (IGF-1 and IGF-2), a family of six high-affinity IGF binding proteins (IGFBP 1-6), as well as associated IGFBP degrading enzymes, referred to collectively as proteases. Insulin-like growth factor 1 (IGF-1) is mainly secreted by the liver as a result of stimulation by growth hormone (GH). IGF-1 is important for both the regulation of normal physiology, as well as a number of pathological states, including cancer. The IGF axis has been shown to play roles in the promotion of cell proliferation and the inhibition of cell death (apoptosis). Insulin-like growth factor 2 (IGF-2) is thought to be a primary growth factor required for early development while IGF-1 expression is required for achieving maximal growth.

EGF also plays a role in the regulation of cell growth, proliferation and differentiation. EGF acts by binding with high affinity to epidermal growth factor receptors (EGFR) on cell surfaces to stimulate the tyrosine kinase activity of the receptors. The tyrosine kinase activity in turn initiates a signal transduction cascade, which results in a variety of biochemical changes within the cell (e.g., increased intracellular calcium levels, increased glycolysis and protein synthesis, and increased expression of the gene for EGFR) that lead to DNA synthesis and cell proliferation.

VEGF is a signaling protein involved in angiogenesis. VEGF has been shown to stimulate endothelial cell mitogenesis and cell migration. VEGF is also a vasodilator and increases microvascular permeability.

Thus, the above growth factors stimulate cell proliferation, differentiation, and migration of cells, and stimulate the growth of blood vessels to supply nutrients to the tissue growth. Apart from the above growth factors, the scaffold 10 may also be coated and/or impregnated with an additional component or components that aid in cell growth and tissue repair. These may be additional growth factors to those listed and discussed above.

Further, the at least one bioactive agent 22 may be an ECM protein. As is known to those of ordinary skill in the art, the ECM is the extracellular part of tissue that is the defining feature of connective tissue in animals, and is composed of an interlocking mesh of fibrous proteins and glycosminoglycans (GAGs). Due to its diverse nature and composition, the ECM can serve many functions, such as providing support and anchorage for cells, segregating tissues from one another, and regulating intercellular communication. Further, it sequesters and contains a wide range of cellular growth factors. When the at least one bioactive agent 22 is an ECM protein, it may be chosen from fibronectin, laminin, collagen, elastin, and aggrecan.

Fibronectin is a high-molecular-weight glycoprotein that binds to membrane spanning receptor proteins called integrins. Fibronectin also binds ECM components such as collagen, fibrin and heparan sulfate. More specifically, fibronectins connect cells with collagen fibers in the ECM, allowing cells to move through the ECM. Fibronectins are secreted by cells in an unfolded, inactive form. Binding to integrins unfolds fibronectin molecules, allowing them to form dimers so that they can function properly. Fibronectins also help at the site of tissue injury by binding to platelets during blood clotting and facilitating cell movement to the affected area during wound healing.

Laminins are proteins found in the ECM that form networks of web-like structures that resist tensile forces in the basal lamina. They also assist in cell adhesion. Laminins bind other ECM components such as collagens, nidogens, and entactins. In particular, laminin is the major noncollagenous component of the basal lamina, such as those on which cells of an epithelium sit. Laminins form independent networks and are associated with type IV collagen networks via entactin, and perlecan. They also bind to cell membranes through integrin receptors and other plasma membrane molecules, such as the dystroglycan glycoprotein complex and Lutheran blood group glycoprotein. Through these interactions, laminins critically contribute to cell attachment and differentiation, cell shape and movement, maintenance of tissue phenotype, and promotion of tissue survival.

Collagens are, in most animals, the most abundant glycoproteins in the ECM. In fact, collagen is the most abundant protein in the human body and accounts for 90% of bone matrix protein content. Collagens are present in the ECM as fibrillar proteins and give structural support to resident cells. Collagen can be divided into several families according to the types of structure: Fibrillar (Type I, II, III, V, XI); Facit (Type IX, XII, XIV); Short chain (Type VIII, X); Basement membrane (Type IV); Other (Type VI, VII, XIII).

Elastins, in contrast to collagens, give elasticity to tissues, allowing them to stretch when needed and then return to their original state. This is useful in blood vessels, the lungs and in skin, and these organs contain high amounts of elastins. Elastins are synthesized by fibroblasts and smooth muscle cells.

Aggrecans, as is known to those of ordinary skill in the art, are another protein found in the ECM having a structural function. Aggrecans are proteoglycans. Along with Type-II collagen, aggrecan forms a major structural component of cartilage.

Thus, the above ECM proteins assist in providing structural support to cells. Apart from the above ECM proteins, the scaffold 10 may also be coated and/or impregnated with an additional component or components that aid in such structural support. These may be additional ECM proteins, such as those listed and discussed above.

Further, the at least one bioactive agent 22 may be a hormone. As is known to those of ordinary skill in the art, a hormone is a chemical messenger that transports a signal from one cell to another. Cells respond to a hormone when they express a specific receptor for that hormone. The hormone binds to the receptor protein, resulting in the activation of a signal transduction mechanism that ultimately leads to cell type-specific responses. When the at least one bioactive agent 22 is a hormone, it may be chosen from parathyroid hormone, growth hormone, insulin, glucagon, and calcitonin.

Parathyroid hormone (PTH) is secreted by the parathyroid glands. It acts to increase the concentration of calcium in the blood, whereas calcitonin, discussed below, (a hormone produced by the parafollicular cells of the thyroid gland) acts to decrease calcium concentration. PTH acts to increase the concentration of calcium in the blood by acting upon parathyroid hormone receptor.

Growth hormone (GH) is a hormone that stimulates growth and cell reproduction in humans and other animals. It is synthesized, stored, and secreted by the somatotroph cells within the anterior pituitary gland. GH directly stimulates division and multiplication of chondrocytes of cartilage.

Insulin is a hormone with extensive effects on both metabolism and several other body systems (e.g., vascular compliance). When present, it causes most of the body's cells to take up glucose from the blood (including liver, muscle, and fat tissue cells), storing it as glycogen in the liver and muscle, and stops use of fat as an energy source.

Glucagon is an important hormone involved in carbohydrate metabolism. Produced by the pancreas, it is released when the glucose level in the blood is low (hypoglycemia), causing the liver to convert stored glycogen into glucose and release it into the bloodstream. The action of glucagon is thus opposite to that of insulin, which instructs the body's cells to take in glucose from the blood in times of satiation.

Calcitonin is produced in humans primarily by the parafollicular (also known as C-cells) of the thyroid. As mentioned above, it acts to reduce blood calcium, opposing the effects of PTH. Its actions, in a broad sense, are bone mineral metabolism by promoting mineralization of skeletal bone and protecting against calcium loss from skeleton during periods of calcium stress, such as pregnancy and lactation.

Apart from the above hormones, the scaffold 10 may also be coated and/or impregnated with an additional component or components that aid in cell growth and tissue repair. These may be additional hormones, such as those listed and discussed above.

Further, the at least one bioactive agent 22 may be a cytokine. Cytokines are a category of signaling proteins and glycoproteins that, like hormones, are used in cellular communication. While hormones are secreted from specific organs to the blood, cytokines are a more diverse class of compounds. They are produced by a wide variety of hematopoietic and nonhematopoietic cell types and can have effects on both nearby cells or throughout the organism, sometimes strongly dependent on the presence of other chemicals. When the at least one bioactive agent 22 is a cytokine, it may be chosen from interferon, interleukin, colony stimulating factor, stem cell factor, erythropoietin, and thrombopoietin.

Interferons are proteins produced by the cells of the immune system in response to challenges by foreign agents such as viruses, parasites, and tumor cells. Interferons assist the immune response by inhibiting viral replication within host cells, activating natural killer cells and macrophages, increasing antigen presentation to lymphocytes, and inducing the resistance of host cells to viral infection.

Interleukins are a group of cytokines (secreted signaling molecules) that were first seen to be expressed by white blood cells (leukocytes, hence the -leukin) as a means of communication. In particular, interleukins are the cytokines that act specifically as mediators between leucocytes. Interleukins regulate cell growth, differentiation and motility.

Colony stimulating factors (CSF) are secreted glycoproteins, which bind to receptor proteins on the surfaces of hemopoietic stem cells and thereby activate intracellular signaling pathways, which can cause the cells to proliferate and differentiate into a specific kind of blood cell (usually white blood cells).

Stem cell factor (SCF) is a cytokine that exists in two forms, cell surface bound SCF and soluble (or free) SCF. SCF is a growth factor important for the survival, proliferation, and differentiation of hematopoietic stem cells and other hematopoietic progenitor cells.

Erythropoietin is a glycoprotein hormone that is a cytokine for erythrocyte (red blood cell) precursors in the bone marrow. It is produced by the liver and kidney, and is the hormone that regulates red blood cell production. It also has other known biological functions, such as assisting in the brain's response to neuronal injury, and is involved in the wound healing process.

Thrombopoietin is a glycoprotein hormone produced mainly by the liver and the kidney that regulates the production of platelets by the bone marrow. It stimulates the production and differentiation of megakaryocytes, the bone marrow cells that fragment into large numbers of platelets.

Apart from the above cytokines, the scaffold 10 may also be coated and/or impregnated with an additional component or components that aid in cell growth and tissue repair. These may be additional cytokines, such as those listed and discussed above.

Finally, the at least one bioactive agent 22 may be a chemokine, Chemokines are a family of small cytokines, or proteins secreted by cells. The major role of chemokines is to guide the migration of cells. Cells that are attracted by chemokines follow a signal of increasing chemokine concentration towards the source of the chemokine. Some chemokines are considered pro-inflammatory and can be induced during an immune response to promote cells of the immune system to a site of infection, while others are considered homeostatic and are involved in controlling the migration of cells during normal processes of tissue maintenance or development. These proteins exert their biological effects by interacting with G protein-linked transmembrane receptors called chemokine receptors, which are selectively found on the surfaces of their target cells. When the at least one bioactive agent 22 is a chemokine, it may be chosen from RANTES, stromal cell derived factor, macrophage inflammatory protein, monocyte chemotactic protein, and interferon-inducible protein.

RANTES (also known as CCL5) is a protein that is chemotactic for T cells, eosinophils, and basophils, and plays an active role in recruiting leukocytes into inflammatory sites. With the help of particular cytokines (i.e., IL-2 and IFN-y) that are released by T cells, RANTES also induces the proliferation and activation of certain natural-killer (NK) cells.

Stomal cell derived factor is small cytokine belonging to the chemokine family that plays an important role in angoigenesis by recruiting endothelial progenitor cells (EPC) from the bone morrow through a CXCR4 dependent mechanism. Stromal cell-derived factors 1-alpha and 1-beta are small cytokines that belong to the intercrine family, members of which activate leukocytes and are often induced by proinflammatory stimuli such as lipopolysaccharide, TNF, or IL1.

Macrophage Inflammatory Proteins (MIP) include two major forms, MIP-1α and MIP-1β (also known as CCL3 and CCL4, respectively). Both are major factors produced by macrophages after they are stimulated with bacterial endotoxins. They activate human granulocytes (neutrophils, eosinophils and basophils) which can lead to acute neutrophilic inflammation. They also induce the synthesis and release of other pro-inflammatory cytokines such as interleukin 1 (IL-1), IL-6 and TNF-α from fibroblasts and macrophages.

Monocyte chemotactic protein (also known as CCL2) is a small cytokine belonging to the CC chemokine family. CCL2 recruits monocytes, memory T cells, and dendritic cells to sites of tissue injury and infection. In human osteoclasts, it has been shown that CCL2 and RANTES (regulated on activation normal T cell expressed and secreted) are unregulated by RANKL (receptor activator of NFκB ligand). Both MCP-1 and RANTES were also shown to induce the formation of TRAP-positive, multinuclear cells from M-CSF-treated monocytes in the absence of RANKL, but produced osteoclasts that lacked cathepsin K expression and resorptive capacity. It is proposed that CCL2 and RANTES act as autocrine loop in human osteoclast differentiation.

Interferon-inducible protein is expressed and secreted by monocytes, fibroblasts, and endothelial cells after stimulation with IFN-γ and has important roles in the migration of T cells into inflamed sites. It also promotes the regression of angiogenesis.

Apart from the above chemokines, the scaffold 10 may also be coated and/or impregnated with an additional component or components that aid in cell growth and tissue repair. These may be additional chemokines, such as those listed and discussed above.

Thus, the various exemplary embodiments illustrated in FIGS. 1A-1E provide a system wherein at least one cell 12 is cultured in the same medium as a scaffold 10, and within the same interior compartment 18 as the scaffold 10, but without contact between the at least one cell 12 and the scaffold 10. Thus, as the at least one cell 12 produces various bioactive agents 22, such as growth factors, the bioactive agents 22 enter the medium 20, and contact and coat and/or impregnate the scaffold 10. However, due to the physical separation of the scaffold 10 and at least one cell 12, the at least one cell 12 (and any cell debris) is prevented from coating and/or impregnating the scaffold 10.

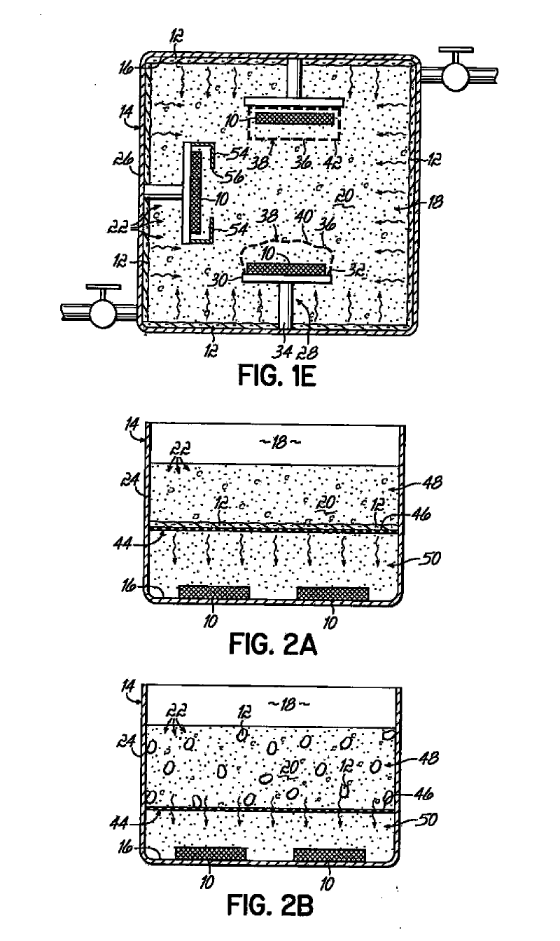

In the embodiments illustrated in FIGS. 2A and 2B, the system may further include structure to create separate areas within the interior compartment 18 of the container 14. This allows the at least one cell 12 to be cultured in the same medium 20 as the scaffold 10, but does not include the scaffold support structure (used and shown in the embodiments of FIGS. 1A-1E). Thus, in the embodiments of FIGS. 2A and 2B, the system includes a bioactive agent permeable support 44 positioned between the at least one cell 12 and the scaffold 10. This bioactive agent permeable support 44 is permeable to the at least one bioactive agent 22 (while not being permeable to the at least one cell 12 or any cell debris), and the scaffold 10 is disposed on a surface 16 of the container 14.

More specifically, the system shown in the embodiment illustrated in FIG. 2A includes a bioactive agent permeable support 44 disposed within the medium 20. In this embodiment, the at least one cell 12 may be an adherent cell, and is disposed on the bioactive agent permeable support 44. However, as will be recognized by those of ordinary skill in the art, the at least one cell 12 does not have to be positioned on or adhered to the bioactive agent permeable support 44. In alternate embodiments (not shown), the at least one cell 12 may be an adherent cell or adherent cells positioned on or adhered to surfaces of the container (such as sidewall surfaces). Further, as can be seen in the illustrated embodiment of FIG. 2A, the bioactive agent permeable support 44 is disposed within the medium 20 and separates the interior compartment 18 of the container 14 into a first area 48 and a second area 50. The scaffold 10 is positioned in the first area 48, and more specifically is positioned on a surface 16 of the container 14. However, the scaffold 10 does not need to be positioned on this particular surface 16, but may alternatively be positioned on other surfaces of the container 14 (such as sidewall surfaces). The at least one cell 12 is positioned within the second area 50, and in the particularly illustrated embodiment, is adhered to the bioactive agent permeable support 44 within the second area 50. As described above, alternatively, the at least one adherent cell 12 may be adhered to a side wall of the container 14 rather than the bioactive agent permeable support 44, or may be adhered to both the bioactive permeable support 44 and the side wall surface 16 of the container 14. The bioactive agent permeable support 44 includes apertures 36 that are sized such that the at least one bioactive agent 22 may pass therethrough from the second chamber to the first chamber within the medium 20, but the at least one cell 12 (and any cell debris) cannot pass therethrough. Thus, as shown in the illustrated embodiment, the bioactive agent 22 may coat and/or impregnate the scaffold 10 while the scaffold 10 remains physically separated from the at least one cell 12.

Like the system shown in FIG. 2A, the system shown in the embodiment illustrated in FIG. 2B also includes a bioactive agent permeable support 44 disposed within the medium 20. In this embodiment, the at least one cell 12 may be an nonadherent cell, and thus forms a cell suspension within the medium 20. Further, as can be seen in the illustrated embodiment of FIG. 2B, the bioactive agent permeable support 44 is disposed within the medium 20 and separates the interior compartment 18 of the container 14 into a first area 48 and a second area 50. The scaffold 10 is positioned in the first area 48, and more specifically is positioned on a surface 16 of the container 14. However, the scaffold 10 does not need to be positioned on this particular surface 16, but may alternatively be positioned on other surfaces of the container 14 (such as sidewall surfaces). The at least one cell 12 is positioned within the second area 50, and in the particularly illustrated embodiment, is within the medium 20 and forms a cell suspension therewith. The bioactive agent permeable support 44 includes apertures 36 that are sized such that the at least one bioactive agent 22 may pass therethrough from the second chamber to the first chamber within the medium 20, but the at least one cell 12 (and any cell debris) cannot pass therethrough. Thus, as shown in the illustrated embodiment, the bioactive agent 22 may coat and/or impregnate the scaffold 10 while the scaffold 10 remains physically separated from the at least one cell 12.

Like the embodiments described above with respect to FIGS. 1A-1E, the at least one bioactive agent 22 in the embodiments of FIGS. 2A and 2B may be chosen from a growth factor (e.g., EMP, TGF, FGF, PDGF, IGF, EGF, VEGF, etc., and combinations thereof), an ECM protein (e.g., fibronectin, laminin, collagen, elastin, aggrecan, etc., and combinations thereof), a hormone (e.g., parathyroid hormone, growth hormone, insulin, glucagon, calcitonin, etc., and combinations thereof), a cytokine (e.g., interferon, interleukin, colony stimulating factor, stem cell factor, erythropoietin, thrombopoietin, etc, and combinations thereof), and a chemokine (e.g., RANTES, stromal cell derived factor, macrophage inflammatory protein, monocyte chemotactic protein, interferon-inducible protein, etc., and combinations thereof).

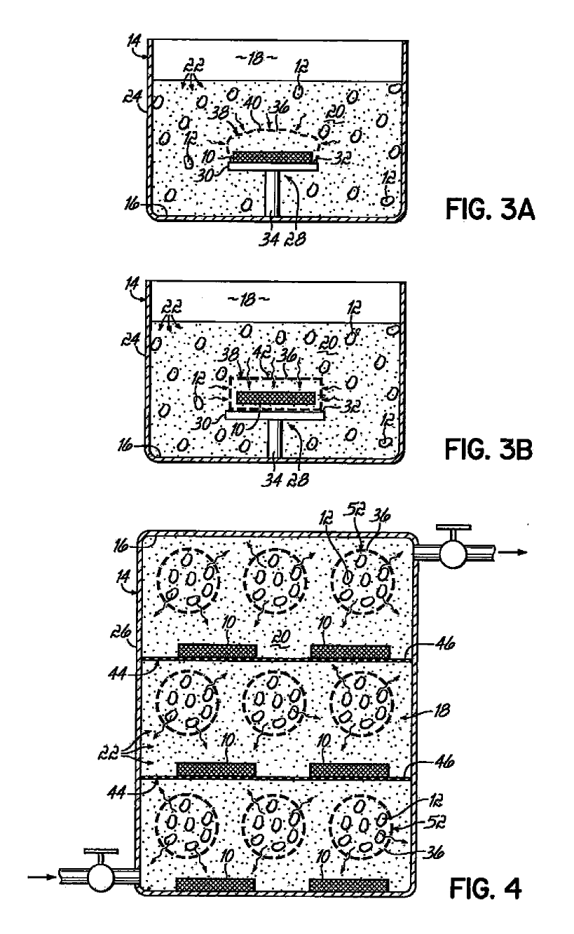

Referring now to FIGS. 3A and 3B, the at least one cell 12 may be a nonadherent cell and is disposed within the medium 20. In such an embodiment, the at least one cell 12 disposed within the medium 20 may form a cell suspension. Thus, the scaffold 10 could be coated and/or impregnated with growth factors secreted by nonadherent cells while the scaffold 10 is isolated in a chamber 42 or membrane bag 40 that is permeable to proteins but not cells and cell debris. For example, and referring to FIG. 3A, one may place the scaffold 10 in a membrane 40 on the scaffold support structure 28. The membrane 40 has many apertures 36 that are permeable to bioactive agents 22, but not to the at least one cell 12 or any cell debris. As described above, the at least one cell 12 can produce different bioactive agents 22, and so, if a specific bioactive agent is desired, a barrier 40 (such as a filter or membrane 40) with apertures 36 having specific diameters corresponding to the molecular weight of that factor, may be used to separate out the undesired bioactive agents 22 (for example, less than 20 kilo Dalton to allow passage of specific bioactive agents). Thus, the membrane 40 can be made of plastic or filter-like membranes. During cell culture, cell secreted growth factors or other desired bioactive agents 22 will be trapped or absorbed on the scaffold 10 in the culture system.

Alternatively, and referring to FIG. 3B, the barrier 38 may be a chamber 42 that surrounds the scaffold 10. Like the membrane 40, the chamber 42 has many apertures 36 that are permeable to bioactive agents 22 of a certain size, but not to the at least one cell 12, or any cell debris.

The embodiment particularly illustrated in FIGS. 3A and 3B further includes a scaffold support structure 28 positioned within the medium 20, the scaffold 10 being disposed on the scaffold support structure 28. In the illustrated embodiments, the scaffold support structure 28 includes a scaffold support 30 having a surface 32 on which the scaffold 10 is disposed, and a post 34. The particular configuration of the scaffold support structure 28 of the figures is merely exemplary, as any configuration that provides a surface for the scaffold while keeping the scaffold physically separated from the at least one cell will suffice. Thus, the scaffold support structure 28 provides a surface 32 for the scaffold 10 other than the surface 16 of the interior compartment 18 of the container 14. The scaffold 10 may then be disposed on the scaffold support structure 28.

However, it will be recognized by those skilled in the art that a scaffold support structure is not necessary when nonadherent cells are used. For example, in an alternative embodiment (not shown), the scaffold may simply be disposed on a surface of the container (and within the medium) as long as it is surrounded by a bioactive agent permeable bather 38.

Like the embodiments described above with respect to FIGS. 1A-2B, the at least one bioactive agent 22 in the embodiments of FIGS. 3A and 3B may be chosen from a growth factor (e.g., BMP, TOE, FGF, PDGF, IGF, EGF, VEGF, etc., and combinations thereof), an ECM protein (e.g., fibronectin, laminin, collagen, elastin, aggrecan, etc., and combinations thereof), a hormone (e.g., parathyroid hormone, growth hormone, insulin, glucagon, calcitonin, etc., and combinations thereof), a cytokine (e.g., interferon, interleukin, colony stimulating factor, stem cell factor, erythropoietin, thrombopoietin, etc., and combinations thereof), and a chemokine (e.g., RANTES, stromal cell derived factor, macrophage inflammatory protein, monocyte chemotactic protein, interferon-inducible protein, etc., and combinations thereof).

Referring now to FIG. 4, the at least one cell 12 may be a nonadherent cell and is disposed within the medium 20, but within a bioactive agent permeable capsule 52. In such an embodiment, the at least one cell 12 disposed within the medium 20 may form a cell suspension (within the bioactive agent permeable capsule 52). Thus, nonadherent cells are captured and cultured in the bioactive agent permeable capsule 52 while scaffolds 10 are placed in the same culture system, (e.g., a bioreactor 26 or a culture vessel 24), so that the desired bioactive agents 22 are released into medium 20 within the bioactive agent permeable capsule 52, pass through the permeable capsule 52 into the medium 20 exterior to the capsule 52, and are then coated onto and/or impregnated into the scaffold 10. In this embodiment, the material of the permeable capsule 52 (or the capsule 52 itself) may be chosen from a chamber 42 and a membrane 40.

Like the embodiments described above with respect to FIGS. 1A-3B, the at least one bioactive agent 22 in the embodiment of FIG. 4 may be chosen from a growth factor (e.g., BMP, TGF, FGF, PDGF, IGF, EGF, VEGF, etc., and combinations thereof), an ECM protein (e.g., fibronectin, laminin, collagen, elastin, aggrecan, etc., and combinations thereof), a hormone (e.g., parathyroid hormone, growth hormone, insulin, glucagon, calcitonin, etc., and combinations thereof), a cytokine (e.g., interferon, interleukin, colony stimulating factor, stem cell factor, erythropoietin, thrombopoietin, etc., and combinations thereof), and a chemokine (e.g., RANTES, stromal cell derived factor, macrophage inflammatory protein, monocyte chemotactic protein, interferon-inducible protein, etc., and combinations thereof).

In addition, bioactive agents 22 that are not coated onto, the scaffold 10 in the system of the present invention may be concentrated by commercially available columns (not shown), such as protein-concentration columns, and then separately coated onto and/or impregnated into the scaffold 10. For example, if the molecular weight of a desired bioactive agent is 20 KD, a filter (column) permeable to bioactive agents having a molecular weight less that 20 KD (e.g., 15-18 KD) could be used to remove bioactive agents and other molecules that are smaller than 20 KD. After this first filtration, the medium including the bioactive agents having a molecular weight greater than or equal to 20 KD could be filtered again using a filter (column) permeable to bioactive agents having a molecular weight greater than 20 KD (e.g., 25-30 KD). The remaining medium then would include bioactive agents having a molecular weight of about 20 KD (or in a range thereof). This bioactive agent-containing medium could then be used to further coat and/or impregnate the scaffold 10 to prepare a scaffold having a greater concentration of bioactive agents, or a scaffold 10 having a large concentration of a particular, desired bioactive agent or agents.

To limit other foreign bioactive agents in the culture system (thereby preventing them from being coated onto and/or impregnated into the scaffold), serum-free medium can be used during the whole cell culture process. Alternatively, serum medium may be used during cell expansion, followed by switching to serum-free medium during the coating of the scaffold.

Referring now to FIG. 5, another aspect of the present invention provides a device for repair of a tissue. The device includes at least one cell-produced bioactive agent 22, and a scaffold 10 having affinity for the at least one cell-produced bioactive agent 22. The scaffold 10 has been cultured in the presence of, but absent direct physical contact with, the at least one cell-produced bioactive agent 22.

Further, like the embodiments described above with respect to FIGS. 1A-4, the at least one bioactive agent 22 in the embodiment of FIG. 5 may be chosen from a growth factor (e.g., BMP, TGF, FGF, PDGF, IGF, EGF, VEGF, etc., and combinations thereof), an ECM protein (e.g., fibronectin, laminin, collagen, elastin, aggrecan, etc., and combinations thereof), a hormone (e.g., parathyroid hormone, growth hormone, insulin, glucagon, calcitonin, etc., and combinations thereof), a cytokine (e.g., interferon, interleukin, colony stimulating factor, stem cell factor, erythropoietin, thrombopoietin, etc., and combinations thereof), and a chemokine (e.g., RANTES, stromal cell derived factor, macrophage inflammatory protein, monocyte chemotactic protein, interferon-inducible protein, etc., and combinations thereof).

Another aspect of the present invention includes a method of coating and/or impregnating a scaffold 10 with at least one bioactive agent 22. The method includes disposing at least one cell 10 capable of producing at least one bioactive agent 22 in medium 20 in a container 14, positioning a scaffold 10 in the medium 20, and maintaining the scaffold 10 physically separated from the at least one cell 12 while culturing the at least one cell 12 in the medium 20.

In one embodiment of the method, disposing the at least one cell 12 in the medium 20 may further include positioning the at least one cell 12 on a surface 16 of the container 14, such that it adheres thereto. Thus, this embodiment includes an adherent cell disposed on a surface 16 of the container 14 as the at least one cell 12. Thus, at least one adherent cell 12 may be cultured on a surface 16 of a container 14, such as the bottom of a cell culture dish or on the wall of a bioreactor 26. During the cell culture, a protein affinitive scaffold is immersed in the medium 20 physically separate from the bottom of the cell culture dish or the wall of the bioreactor 26 in multiple ways. For example, the method may further include positioning the scaffold 10 on a scaffold support structure 28 disposed within the medium 20. The scaffold support structure 28 provides a surface 46 for the scaffold 10 other than the actual surface formed by the interior compartment 18 of the container 14. The scaffold 10 may then be disposed on the scaffold support structure 28. In this manner, the scaffold 10 is kept physically separated from the at least one cell 12.

The method may further include positioning the scaffold 10 within a bather 44. In one such embodiment, the barrier 38 surrounds the scaffold 10. In that embodiment, the bather 38 may be chosen from a chamber 42 and a membrane 40. For example, one may place the scaffold 10 in a chamber 42 on a rack that is away from cells. The chamber 42 has apertures 36 that are permeable to proteins at a certain size, but not to cell debris, and the chamber 42 can be made of metal, plastic or filter-like membranes. During cell culture, cell secreted growth factors or desired proteins or other bioactive agents will be trapped or absorbed on the bioactive agent-affinitive scaffold in the culture system.

Further, disposing the at least one cell 12 in the medium 20 may further include positioning the at least one cell 12 on a bioactive agent permeable support 44 disposed within the medium 20. The bioactive agent permeable support 44 is disposed within the medium 20 and separates the interior compartment 18 of the container 14 into a first area 48 and a second area 50. The scaffold 10 is positioned on a surface 16 of the container 14 in the first area 48, and the at least one cell 12 is adhered to the bioactive agent permeable support 44 within the second area 50. The bioactive agent permeable support 44 includes apertures 36 that are sized such that the at least one bioactive agent 22 may pass therethrough from the second chamber 50 to the first chamber 48 within the medium 20, but the at least one cell 12 cannot pass therethrough. Thus, in this embodiment of the method, the bioactive agent 22 may coat and/or impregnate the scaffold 10 while the scaffold 10 remains physically separated from the at least one cell 12.

In other embodiments of the method, the at least one cell 12 may be a nonadherent cell and is disposed within the medium 20. In such an embodiment, the at least one cell 12 disposed within the medium 20 may form a cell suspension. Thus, the bioactive agent permeable support 44 is disposed within the medium 20 and separates the interior compartment 18 of the container 14 into a first area 48 and a second area 50. The non adherent cells float within the medium 20, thereby forming a cell suspension, in the second area 50 of the interior compartment 18 of the container 14. The bioactive agent permeable support 44 includes apertures 36 that are sized such that the at least one bioactive agent 22 may pass therethrough from the second chamber 50 to the first chamber 48 within the medium 20, but the at least one cell 12 cannot pass therethrough.

The method may further include positioning the at least one cell 12 within a bioactive agent permeable capsule 52. In such an embodiment, the at least one cell 12 disposed within the medium 20 may form a cell suspension (but only within the bioactive agent permeable capsule 52. Thus, nonadherent cells are captured and cultured in the bioactive agent permeable capsule 52 while scaffolds 10 are placed in the same culture system, (e.g., a bioreactor 26 or a culture vessel 24), so that the desired bioactive agents 22 are released into medium 20 within the bioactive agent permeable capsule 52, pass through the permeable capsule 52 into the medium 20 exterior to the capsule 52, and are then coated onto or impregnated into the scaffold 10. In this embodiment, the material of the permeable capsule 52 (or the capsule 52 itself) may be chosen from a chamber 42 and a membrane 40.

While the invention has been illustrated by the description of various embodiments, and while the various embodiments have been described in considerable detail, the inventors do not intend to restrict or in any way limit the scope of the appended claims to such detail. Additional advantages and modifications will readily appear to those skilled in the art. Moreover, the various features of the invention may be used alone or in numerous combinations depending on the needs and preferences of the user. Therefore, the invention in its broader aspects is not limited to the specific details, representative apparatus and methods, and illustrative examples shown and described. Accordingly, departures may be made from such details without departing from the scope or spirit of the general inventive concept.

1. A system for producing a scaffold coated and/or impregnated with at least one bioactive agent, comprising:
a container having a surface defining an interior compartment, wherein a medium is disposed within the interior compartment;
at least one cell disposed within the medium, the at least one cell capable of producing at least one bioactive agent; and
a scaffold disposed within the medium, the scaffold being physically separated from the at least one cell such that there is no contact between the scaffold and the at least one cell.

2. The system of claim 1, wherein the container is chosen from a cell culture vessel and a bioreactor.

3. The system of claim 1, wherein the at least one cell is an adherent cell and is disposed on the surface of the container.

4. The system of claim 1, further comprising a bioactive agent permeable support disposed within the medium.

5. The system of claim 4, wherein the at least one cell is an adherent cell, and is disposed on the bioactive agent permeable support.

6. The system of claim 1, wherein the at least one cell is a nonadherent cell and is disposed within the medium.

7. The system of claim 6, wherein the at least one cell disposed within the medium forms a cell suspension.

8. The system of claim 1, further comprising a scaffold support structure positioned within the medium, the scaffold being disposed on the scaffold support structure.

9. The system of claim 8, wherein the scaffold support structure is permeable to the at least one bioactive agent.

10. The system of claim 1, further comprising a bather between the at least one cell and the scaffold, the barrier being permeable to the at least one bioactive agent.

11. The system of claim 10, wherein the barrier surrounds the scaffold.

12. The system of claim 11, wherein the barrier is chosen from a chamber and a membrane.

13. The system of claim 10, wherein the scaffold is disposed on a surface of the container.

14. The system of claim 13, wherein the barrier surrounds the scaffold, and wherein the barrier is chosen from a chamber and a membrane.

15. The system of claim 13, further comprising a bioactive agent permeable support disposed within the medium and separating the interior compartment into a first area and a second area, wherein the scaffold is disposed within the first area and the at least one cell is disposed within the second area.

16. The system of claim 10, wherein the barrier surrounds the at least one cell.

17. The system of claim 16, wherein the barrier is chosen from a chamber and a membrane.

18. The system of claim 1, wherein the medium is a serum-free cell culture medium.

19. The system of claim 1, wherein the at least one bioactive agent is chosen from a growth factor, an extracellular matrix protein, a hormone, a cytokine, and a chemokine.

20. The system of claim 19, wherein the at least one bioactive agent is a growth factor chosen from BMP, TGF, FGF, PDGF, IGF, EGF, and VEGF.

21. The system of claim 19, wherein the at least one bioactive agent is an extracellular matrix protein chosen from fibronectin, laminin, collagen, elastin and aggrecan.

22. The system of claim 19, wherein the at least one bioactive agent is a hormone chosen from parathyroid hormone, growth hormone, insulin, glucagon, and calcitonin.

23. The system of claim 19, wherein the at least one bioactive agent is a cytokine chosen from interferon, interleukin, colony stimulating factor, stem cell factor, erythropoietin, and thrombopoietin.

24. The system of claim 19, wherein the at least one bioactive agent is a chemokine chosen from RANTES, stromal cell derived factor, macrophage inflammatory protein, monocyte chemotactic protein, and interferon-inducible protein.

25. A method of coating and/or impregnating a scaffold with at least one bioactive agent, comprising:
disposing at least one cell capable of producing at least one bioactive agent in medium in a container,
positioning a scaffold in the medium; and
maintaining the scaffold physically separated from the at least one cell while culturing the at least one cell in the medium.

26. The method of claim 25, wherein disposing the at least one cell in the medium further comprises positioning the at least one cell on a surface of the container, such that it adheres thereto.

27. The method of claim 25, wherein disposing the at least one cell in the medium further comprises positioning the at least one cell on a bioactive agent permeable support disposed within the medium.

28. The method of claim 25, wherein disposing the at least one cell in the medium further comprises suspending the at least one cell in the medium.

29. The method of claim 25, wherein the medium is a cell culture medium including serum, and further comprising removing the cell culture medium including serum following cell expansion.

30. The method of claim 29, further comprising adding a serum free cell culture medium to the container following cell expansion.

31. The method of claim 25, wherein positioning the scaffold in the medium further comprises positioning the scaffold on a surface of the container.

32. The method of claim 31, further comprising adhering the scaffold to the surface.

33. The method of claim 25, further comprising positioning the scaffold on a scaffold support structure disposed within the medium.

34. The method of claim 25, further comprising positioning the scaffold within a bioactive agent permeable bather.

35. The method of claim 25, further comprising positioning the at least one cell within a bioactive agent permeable bather.

36. The method of claim 25, wherein the container includes a bioactive agent permeable bather dividing the container into a first compartment and a second compartment, and further comprising disposing the at least one cell in the first compartment and positioning the scaffold in the second compartment.

37. A device for repair of a tissue, comprising:
at least one cell-produced bioactive agent; and
a scaffold having affinity for the at least one cell-produced bioactive agent;
wherein the scaffold has been cultured in the presence of, but absent direct physical contact with, the at least one cell-produced bioactive agent.

38. The device of claim 37, wherein the at least one cell-produced bioactive agent is chosen from a growth factor, an extracellular matrix protein, a hormone, a cytokine, and a chemokine.

39. The device of claim 38, wherein the at least one cell produced bioactive agent is a growth factor chosen from BMP, TGF, FGF, PDGF, IGF, EGF, and VEGF.

40. The device of claim 38, wherein the at least one cell-produced bioactive agent is an extracellular matrix protein chosen from fibronectin, laminin, collagen, elastin, and aggrecan.

41. The device of claim 38, wherein the at least one cell-produced bioactive agent is a hormone chosen from parathyroid hormone, growth hormone, insulin, glucagon, and calcitonin.

42. The device of claim 38, wherein the at least one bioactive agent is a cytokine chosen from interferon, interleukin, colony stimulating factor, stem cell factor, erythropoietin, and thrombopoietin.

43. The device of claim 39, wherein the at least one bioactive agent is a chemokine chosen from RANTES, stromal cell derived factor, macrophage inflammatory protein, monocyte chemotactic protein, and interferon-inducible protein.
